(19) United States
(12) Patent Application Publication
(10) Pub. No.: US 2015/0190269 A1
(43) Pub. Date: Jul. 9, 2015

(54) Pressure Material

(71) Applicants: Bertrand Louis Julien Lenoble, Silly (BE); Elizabeth F. Mallen, Dinas Powis (GB); Steven Robson, Cowbridge (GB); Graham D. Budden, Bridgend (GB)

(72) Inventors: Bertrand Louis Julien Lenoble, Silly (BE); Elizabeth F. Mallen, Dinas Powis (GB); Steven Robson, Cowbridge (GB); Graham D. Budden, Bridgend (GB)

(73) Assignee: DOW CORNING CORPORATION, Midland, MI (US)

(21) Appl. No.: 14/125,136

(22) PCT Filed: Jun. 12, 2012
(86) PCT No.: PCT/EP2012/061107

§ 371 Date: Dec. 10, 2013

(30) Foreign Application Priority Data
Jun. 14, 2011 (EP) 1109949.6

(51) Int. Cl. A61F 5/30; A61F 13/06

(52) U.S. Cl. CPC A61F 5/30; A61F 13/067

(57) ABSTRACT

A method of spreading constant or repeated pressure away from one or more pressure points between a user and an item of wear wherein the item of wear comprises a pressure material being either: (i) a thermoplastic material having the composition comprising a mixture of; (a) component (A) an organic thermoplastic elastomer having a hardness below 80 shore A measured at 23° C. (ISO 868); and (b) component (B) which is a non cross-linked and substantially non reactive silicone polymer or a cross-linked silicone polymer, with the exclusion of borated silicone polymers exhibiting dilatant properties; or (ii) a flexible material having the composition comprising a mixture of; (c) an elastomeric material having a modulus at 100% elongation of 0.1-10 MPa; and (d) 5-80% by weight, based on the total weight of the composition of a non-reactive silicone fluid having a viscosity of 1,000-3,000,000 mPas at 25° C.; or (iii) both (i) and (ii). The use of such a pressure material in the item of wear has been found to provide an excellent method of spreading pressure away from pressure points on the item of wear.

This invention relates to the new use of a material as a pressure spreading material in order to spread pressure from pressure points between a user and an item of wear, particularly but not exclusively an item of footwear. The invention also concerns use of the material in the same manner, as well as articles of manufacture, including a shoe inlay and shoe, including at least in part, such a material.

The term ‘pressure point’ is used in the medical and non-medical fields to relate to an area where the force (generally caused by weight) of a user is constantly or regularly pressurised against a material surface. Non-relief or non-treatment generally leads to inflamed skin, and then sometimes onto well-known subsequent complications. For example, most shoes are generally intended by wearers to be relatively tight fitting, but the tight fitting nature of the shoe can cause the weight of the user to be particularly passed through one or more areas of particular pressure into the shoes, usually via the sole, in use, which will be sore if the correct is ‘ill-fitting’, etc. Other parts of tight fitting shoes may also ‘rub’ during use, against particular parts of a wearer's foot.

Where there is constant pressure, often along with constant rubbing, such pressure points can easily lead to blisters and the like, and easily lead to further complications. This is a particular problem with diabetes sufferers, whose loss of sensitively in their feet can lead to infected blisters occurring without their knowledge, sometimes to such an extent to require lower limb amputation.

Pressure points also occur where constant pressure is applied by inactivity or non-movement of the user against a material, such as lying in bed or for long periods in a chair, without any, or any significant, movement of the user. Again, such constant pressure against the bed or chair can lead to inflamed skin and sores, sometimes termed ‘bedsores’. Bedsores and the like can be caused by pressure based on the compression of tissues, commonly by the force of a bone against a surface, as well as shear forces and possibly friction.

There is a need in the art for a material to relieve pressure from one or more pressure points between a user and an item of wear.

Accordingly, the present invention in one aspect concerns a method of spreading constant or repeated pressure away from one or more pressure points between a user and an item of wear wherein the item of wear comprises a pressure material being either:
(i) a thermoplastic material having the composition comprising a mixture of;
   (a) component (A) an organic thermoplastic elastomer having a hardness below 80 shore A measured at 23° C. (ISO 868); and
   (b) component (B) which is a non cross-linked and substantially non reactive silicone polymer or a cross-linked silicone polymer, with the exclusion of borated silicone polymers exhibiting dilatant properties; or
(ii) a flexible material having the composition comprising a mixture of;
   (c) an elastomeric material having a modulus at 100% elongation of 0.1-10 MPa; and
   (d) 5-80% by weight, based on the total weight of the composition of a non-reactive silicone fluid having a viscosity of 1,000-3,000,000 mPa at 25° C.; or
(iii) both (i) and (ii).

The use of such a pressure material in the item of wear has been found to provide an excellent method of spreading pressure away from pressure points on the item of wear.

The term “constant pressure” as used herein relates to situations involving little or no movement of the relevant parts of a user in relation to the item of wear, generally where they engage, such that pressure caused by the weight of the user remains wholly or substantially constant through the same pressure point(s).

Such situations can include users lying in bed, such as users in a hospital or in other care or long term respite or recovery situations, optionally in a prone or semi-prone position, which users could be intentionally or unintentionally wholly or substantially immobile, or could otherwise be movable infrequently.

Such users can be medical patients, or otherwise incapacitated patients, or elderly users, commonly but not exclusively unable to move themselves significantly. Often, such users lie in a prone or semi-prone position, wherein the pressure points are unrelieved for a period of time. Bedsores or Stage I pressure ulcers can result, sometimes leading to further Stages of damage extending deeper into a user's body.

In another situation, the user is in a sitting position for an extended period, such as in aircraft or other vehicle seat, generally in an expected single position for a lengthy period of time. Whilst some movement of the user may be possible, the weight of the user is often returned to the seat through the same pressure points.

Another example is a situation requiring relatively constant standing by a user in the same or similar position, such as in military guard or patrol police situations, wherein stationery standing position is usually required for lengthy periods of time.

The term “repeated pressure” as used herein relates to situations involving the repeated application of pressure from a user, generally through the user's weight, through the same area of skin to an item of wear. Such pressure may be regular or irregular, and have any frequency, whilst generally resulting in pressure still being applied through the same one or more pressure points.

A first example of repeated pressure may be caused through user's feet, whether this be for walking, running or other foot movements, but wherein the weight of the user is generally passed through to the shoe through the same areas of skin. This is especially where such areas of skin are not those regularly expected to transmit weight, or for which the shoe is expected to receive pressure, especially based on irregular shaped feet, a foot illness or injury, the gait of the user, or the excess pressure caused by a sports or otherwise athlete use of the shoe.

Another example of repeated pressure may be sports or athlete clothing being used during repeated movement or exercise of a user thereagainst.

The method of the present invention is not limited by the nature of the constant or repeated pressure, the frequency or infrequency of any such repeated pressure, and any minor or de minimus changes in pressure, where pressure is then applied again through the same one or more pressure points between the user and item of wear.

The term “pressure point” is as defined above, and includes any area of a user's body through which pressure, generally based on the user's weight, is applied to an item of wear. Such pressure is usually in relation to a matter of ‘comfort’ between the user and an item of wear. Examples of pressure points in a foot for example include the heel, sole, metatarsals and toes (or phalanges). Many foot problems occur from an alteration in the foot bone structure, and metatarsal pain is a common foot problem.

Thus, according to one embodiment of the present invention, the method comprises spreading constant or repeated pressure away from at least two pressure points. Preferably, the at least two pressure points are in a foot. More preferably, at least one pressure point is or involves a metatarsal.

According to another embodiment, the present invention extends to a method of treating one or more of the group comprising;

metatarsalgia; sesamoiditis/fractures; diabetes; stress fractures; shin splints; knee pain; gout; OA; heel spurs; supinated/pes cavus foot types resulting in high plantar pressures; and plantar ulcer, (where there may be some ischaemia caused by circulatory impairment):
by the use of a pressure material as defined herein, preferably by use of the pressure material in or relating to the shoe or shoes of the person with such medical condition.

The method comprises locating an effective amount of the pressure material in or relating to the shoe or shoes of the person with such medical condition(s).

The term “user” as used herein relates to any human or animal.

The term “item of wear” as used herein relates to any article of manufacture, including stand alone items, generally being provided separate such as shoe inlays, or items intended to be used in conjunction with one or more other items, for example materials used for covering beds, chairs and other items of furniture or user support.

According to one embodiment of the present invention, an item of wear comprises one or more of the group comprising: shoes, shoe inlays, sheets, bed covers, seat covers and clothing.

According to a preferred embodiment of the present invention, the pressure material is laminated with one or more other wear layers, such as, but not limited to, one or more harder-wearing outer layers.

Optionally, the pressure material is in the form of a sheet. Optionally, the pressure material is foamed.

Optionally, the pressure material is a thermoplastic material and component (B) comprises:
i. a silicone fluid with a Brookfield viscosity from 1,000 mPa·s to 3,000,000 of mPas at 25° C. (all viscosities, where possible, unless otherwise mentioned, are measured by Brookfield Rotational Viscometer, Model DVIII, Spindle CP52 at 0.5 rpm at 25° C.);
ii. a silicone gum with a molecular weight from 50,000 g/mol to 700,000 g/mol, or
iii. a silicone preparation as liquid silicone rubber or high consistency rubber with no cross-linker and catalyst.

According to another aspect of the present invention, there is provided a shoe inlay comprising a pressure material as defined herein, able to spread constant or repeated pressure away from one or more pressure points between a user and the inlay.

According to another aspect of the present invention, there is provided a shoe comprising a pressure material as defined herein, able to spread constant or repeated pressure away from one or more pressure points between a user and the shoe.

The shoe inlay or a shoe is preferably able to spread constant or repeated pressure away from at least two pressure points.

The shoe inlay or shoe is preferably able to spread constant or repeated pressure away from at least a pressure point being a metatarsal.

According to another embodiment, the pressure material is a thermoplastic (TP) material as defined herein before. Such materials are defined in our WO2010/072882 A1 published on 1 Jul. 2010, and incorporated herein by way of reference.

### Description of Component (A)

Component (A) may be any type of organic thermoplastic elastomer having a hardness below 80 shore A measured at 23° C. according ISO 868. All types of organic thermoplastic elastomer with the respective hardness value can be used. For instance, component (A) can be chosen from the thermoplastic materials cited in Norme ISO 18604:2003, for instance polyamide thermoplastic elastomers, comprising a block copolymer of alternating hard and soft segments with amide chemical linkages in the hard blocks and ether and/or ester linkages in the soft blocks, copolyester thermoplastic elastomers where the linkages in the main chain between the hard and soft segments are chemical linkages being ester and/or ether, olefinic thermoplastic elastomers consisting of a blend of polyolefin and conventional rubber, the rubber phase having little or no cross-linking, styrenic thermoplastic elastomers consisting of at least a triblock copolymer of styrene and a specific diene, where the two end-blocks are polystyrene and the internal block(s) are polydiene or hydrogenated polydiene, urethane thermoplastic elastomers having urethane chemical linkages in the hard blocks and ether, ester or carbonate linkages or mixtures of them in the soft blocks, thermoplastic rubber vulcanisate consisting of a blend of thermoplastic materials and a conventional rubber in which the rubber has been crosslinked by the process of dynamic vulcanisation during the blending and mixing step and mixtures of two or more of these. In particular, the styrene-based elastomers are the preferred ones, alongside thermoplastic polyurethane elastomers. The thermoplastic elastomers with the respective shore hardness values used in EP1060217, EP 1305367, EP1354003 and, in particular EP 1440122 in connection with the polyurethane materials, can be used.

For the avoidance of doubt hard blocks are so named because they have a glass transition point (tg) at a significantly higher temperature than the soft blocks. Typically the hard blocks will have a tg of >50° C. and preferably >80° C. and the soft blocks will have a tg<50° C. typically between −10 and 25° C.

Among organic thermoplastic elastomer having a hardness below 80 shore A measured at 23° C. according ISO 868, there are particularly preferred block copolymers having two or more hard blocks of aromatic vinyl units and one or more unsaturated, partially saturated, or fully saturated aliphatic soft blocks. Preferably, component (A) is a block copolymer having a numerical molecular weight between 30000 g/mol and 500000 g/mol composed of 2 or more hard blocks of aromatic vinyl units having a numerical molecular weight between 2000 g/mol and 70000 g/mol, and one or more unsaturated, partially saturated or fully saturated aliphatic soft blocks. The numerical molecular weight is measured using ASTM D5296-05 and is calculated as polystyrene molecular weight equivalents. Advantageously, the 2 or more hard blocks of aromatic vinyl units have a numerical molecular weight between 2000 g/mol and 70000 g/mol. The most common aromatic vinyl unit is styrene. So, component (A) is, preferably, a styrenic block copolymer having one or more unsaturated, partially saturated or fully saturated aliphatic soft blocks. Styrenic triblock copolymers are preferred.

Into the styrene block copolymers, the most used are the styrenic triblock copolymers known under a normalized nomenclature, as S(B)S, S(I)S, S(EB)S, S(EP)S, S(EEP)S, S(IB)S where S=Styrene, I=isoprene, B=butadiene, EB=ethylene-butylene, EP=ethylene-propylene, EEB=ethylene-ethylene-propylene, IS=isobutylene. The preparation of these block copolymers is well known by the man skilled in the art.

In this invention, styrene block copolymers exhibiting a primary peak of tg (delta) in the temperature range from −10° C. to 50° C. are preferred with high vinyl S(EP)S and S(IB)S are particularly preferred, because they exhibits a primary peak of tg (delta) higher than the others in the temperature range from 0° C. to 50° C. Advantageously, component (A) has its primary tg (delta) loss ratio not below 0.3 between 0° C. and 50° C. (measured dynamic rheometer (Metravib DMA 150) in tensile mode, frequency 10 HZ).

Component (A) may be modified with a hydrocarbon resin miscible with the soft blocks. For instance, component (A) can be formulated with aromatic or aliphatic hydrocarbons resins as C9, C9 hydrogenated, C9 partially hydrogenated, C5, C5/C9 copolymers, terpenes, stabilized rosin ester, dicyclopentadiene (DCPD) hydrogenated to adjust its primary peak of tg(delta) toward the most suitable value and location in temperature for the application.

Apart from hydrocarbon resins, any ingredients used with these copolymers and known in the art, can be added to component (A). Among the ingredients are the plasticizers as those commonly used in the art, such as paraffinic or naphtenic organic oils, organic polymers as polyolefins, mineral fillers and an additive package.

### Description of Component (B)

In the compositions used according to the invention, component (B) can be a non cross-linked silicone or a cross-linked silicone. Nevertheless, component (B) is not a silicone polymer exhibiting dilatant properties in its own right, such as borated silicone polymers as described in WO 03/022085, WO 03/055339 or WO 2005/000966. In other words, the non cross-linked silicone used, for instance in the form of a gum or preparation of gum and silica, is pseudoplastic or shear thinning whereas dilatants are shear thickening. In other words, the dilatant material, in the absence of external force, will be flexible and may even flow, whereas under the effect of impact, it will become temporarily rigid, returning to its flexible state after the impact.

The non cross-linked silicone of component (B) is a substantially non-reactive silicone with respect to component (A) in the absence of a cure package. By “non-reactive” it is meant that it does not react chemically (to form a covalent bond) with precursors of component A. However, preferably the same component (B) can be cross-linked in the presence of a suitable cross-linking package. Preferably the non cross-linked silicone, in the absence of a cross-linking package, is totally non-reactive, but small amounts of some reactivity can be tolerated, e.g. 0.001 to 2 percent. So, when the non cross-linked silicone is non-reactive the composition does not include a cross-linking package, such as a combination of polyorganohydrogensiloxane and hydrosilylation catalyst, as explained hereafter.

When it is a non cross-linked silicone, component (B) can be:
i) a silicone fluid with a Brookfield viscosity from 1,000 mPa·s to 3,000,000 of mPas at 25° C. (all viscosities, where possible, unless otherwise mentioned, are measured by Brookfield Rotational Viscometer, Model DVIII, Spindle CP52 at 0.5 rpm at 25° C.);
ii) a silicone gum with a molecular weight from 50,000 g/mol to 700,000 g/mol, or
iii) a silicone preparation as liquid silicone rubber (LSR) or high consistency rubber (HCR) with no cross-linker and catalyst.
   LSR and HCR are described in more detail below.

When it is cross-linked, component (B) can be formed from the reaction product of (a) a cross-linkable polydiorganosiloxane, (b) optionally a filler and (c) a cross-linking package. A cross-linkable polydiorganosiloxane polymer (a) has at least at least one alkenyl or alkynyl group per end group and optionally alkenyl or alkynyl groups linked to silicon atoms along the polymer backbone. For instance, component (B) can be obtained with i), ii), iii) previously described with the condition that i), ii), iii) contains polydiorganosiloxane alkenyl or alkynyl functional groups in the molecule. A preferred component (B) is a diorganopolysiloxane having a Williams plasticity of at least 30 as determined by ASTM test method 926 and having an average of at least 2 alkenyl radicals in its molecule. Indeed, when component (B) is cross-linked, cross-linking of i), ii), iii) is possible only with a cross-linking package (c) or cross-linking agent: advantageously, polyorganohydrogensiloxane and a hydrosilylation catalyst are added to i), ii), iii). Advantageously, the polyorganohydrogensiloxane contains at least three Si—H groups per molecule.

When component (B) is cross-linked, as it is preferred that component (A) and (B) would be intimately mixed, it is advantageous that cross-linking reaction of the silicone takes place during the hot mixing of component (A) and component (B). Such a process is called dynamic vulcanization and is reported in U.S. Pat. No. 6,013,715, included by reference. Preferably, the mixing and cross linking steps are conducted in a twin-screw extruder. Any suitable process may be utilised but typically component (A) is initially mixed with the polymer of component (B) until good inter-mixing is achieved. The cross-linker is then added followed by further mixing to disperse the cross-linker prior to the introduction of catalyst (if required).

Component (B) is present in the composition in an amount of from 5% to 70% by weight, based on the total weight of the composition. However preferably component (B) is present in an amount of at least 10% by weight, more preferably at least 15% by weight.

### Description LSR

In case component (B) is or is obtained with a liquid silicone rubber (LSR), such LSR can include a polyorganosiloxane polymer which comprises one or more polymers having the formula:

R_{(3-z)}R¹_{z}SiO[R₂SiO)ₓ(RR¹SiO)_{y}]SiR_{(3-z)}R¹_{z}

wherein each R is the same or different and represents a C₁₋₆ alkyl group, an aryl (e.g. phenyl or naphthyl) group or a fluoro-C₁₋₅ alkyl group, preferably each R group is a methyl or ethyl group; R¹ is a C₂₋₆ alkenyl group or an alkynyl group, preferably a vinyl or hexenyl group; x is an integer and y is zero or an integer and x+y is a number (e.g. 100-1000) such that the polymer has a Brookfield viscosity at 25° C. of 50-250,000 mPas, preferably 100-100,000 mPas.

### Description HCR

In the case component (B) is or is obtained with a high consistency rubber (HCR), such HCR can include a polyorganosiloxane polymer which is based on the same formula but the starting Brookfield viscosity of the polymer is greater than 250,000 mPas at 25° C., more usually greater than 500,000 mPas at 25° C. and typically greater than 1,000,000 mPas at 25° C. The upper limit may be many millions. There is nothing preventing the use of a polydiorganosiloxane polymer with a Brookfield viscosity below 250,000 mPas at 25° C. in the present invention, but these would be an LSR rather than a HCR.

Because an HCR is usually in the form of a gum-like material which has such high Brookfield viscosity that the measurement of Brookfield viscosity is extremely difficult, HCRs are often referred by reference to their Williams plasticity number (ASTM D926). The Williams plasticity number of high viscosity polysiloxane gum-like polymers is generally at least 30, typically it is in the range of from about 30 to 250. The plasticity number, as used herein, is defined as the thickness in millimetres×100 of a cylindrical test specimen 2 cm³ in volume and approximately 10 mm in height after the specimen has been subjected to a compressive load of 49 Newtons for three minutes at 25° C. These polysiloxane gum-like polymers generally contain a substantially siloxane backbone (—Si—O—) to which are linked mainly alkyl groups such as for example methyl, ethyl, propyl, isopropyl and t-butyl groups, and some unsaturated groups for example alkenyl groups such as allyl, 1-propenyl, isopropenyl, or hexenyl groups but vinyl groups are particularly preferred and/or combinations of vinyl groups and hydroxyl groups to assist in their cross-linking. Such polysiloxane gum-like polymers typically have a degree of polymerisation (DP) of 500-20,000, which represents the number of repeating Si—O units in the polymer.

The HCR can be a polydiorganosiloxane (this form is particularly adapted for being cross-linked with a cross-linking package) in the form of a polymer or copolymer which contains at least 2 alkenyl radicals having 2 to 20 carbon atoms in its molecule. The alkenyl group is specifically exemplified by vinyl, allyl, butenyl, pentenyl, hexenyl and decenyl. The position of the alkenyl functionality is not critical and it may be bonded at the molecular chain terminals, in non-terminal positions on the molecular chain or at both positions. It is preferred that the alkenyl group is vinyl or hexenyl and that this group is present at a level of 0.001 to 3 weight percent, preferably 0.01 to 1 weight percent, in the polydiorganosiloxane gum.

The remaining (i.e., non-alkenyl) silicon-bonded organic groups in the polydiorganosiloxane are independently selected from hydrocarbon or halogenated hydrocarbon groups which contain no aliphatic unsaturation. These may be specifically exemplified by alkyl groups having 1 to 20 carbon atoms, such as methyl, ethyl, propyl, butyl, pentyl and hexyl; cycloalkyl groups, such as cyclohexyl and cycloheptyl; aryl groups having 6 to 12 carbon atoms, such as phenyl, tolyl and xylyl; aralkyl groups having 7 to 20 carbon atoms, such as benzyl and phenethyl; and halogenated alkyl groups having 1 to 20 carbon atoms, such as 3,3,3-trifluoropropyl and chloromethyl. It will be understood, of course, that these groups are selected such that the polydiorganosiloxane gum has a glass temperature (or melt point) which is below room temperature and the gum is therefore elastomeric. Methyl preferably makes up at least 85, more preferably at least 90, mole percent of the non-unsaturated silicon-bonded organic groups in the polydiorganosiloxane.

Thus, the polydiorganosiloxane can be a homopolymer, a copolymer or a terpolymer containing such organic groups. Examples include gums comprising dimethylsiloxy units and phenylmethylsiloxy units; dimethylsiloxy units and diphenylsiloxy units; and dimethylsiloxy units, diphenylsiloxy units and phenylmethylsiloxy units, among others. The molecular structure is also not critical and is exemplified by straight-chain and partially branched straight-chain, linear structures being preferred.

Specific illustrations of such polydiorganosiloxanes include: trimethylsiloxy-endblocked dimethylsiloxane-methylvinylsiloxane copolymers; trimethylsiloxy-endblocked methylphenylsiloxane-dimethylsiloxane-methylvinylsiloxane copolymers; dimethylvinylsiloxy-endblocked dimethylpolysiloxanes; dimethylvinylsiloxy-endblocked dimethylsiloxane-methylvinylsiloxane copolymers; dimethylvinylsiloxy-endblocked methylphenylpolysiloxanes; dimethylvinylsiloxy-endblocked methylphenylsiloxane-dimethylsiloxane-methylvinylsiloxane copolymers; and similar copolymers wherein at least one end group is dimethylhydroxysiloxy.

Component (iii) HCR may also consist of combinations of two or more polydiorganosiloxanes. Most preferably, component (iii) HCR is a polydimethylsiloxane homopolymer which is terminated with a vinyl group at each end of its molecule or is such a homopolymer which also contains at least one vinyl group along its main chain.

In an embodiment of the present invention, the molecular weight of the polydiorganosiloxane gum is sufficient to impart a Williams plasticity number of at least about 30 as determined by the American Society for Testing and Materials (ASTM) test method 926.

Although there is no absolute upper limit on the plasticity of component (iii) HCR, practical considerations of processability in conventional mixing equipment generally restrict this value. Preferably, the plasticity number should be about 100 to 200, most preferably about 120 to 185.

Methods for preparing high consistency unsaturated group-containing polydiorganosiloxanes are well known and they do not require a detailed discussion in this specification. For example, a typical method for preparing an alkenyl-functional polymer comprises the base-catalyzed equilibration of cyclic and/or linear polydiorganosiloxanes in the presence of similar alkenyl-functional species.

### Cross-Linking of Component (B) by Hydrosilylation

Component (B) can be cross-linked. A cross-linked siloxane polymer can be obtained by reaction of a polydiorganosiloxane (i), (ii) or (iii) with a cross-linking package (c) consisting of a polyorganohydrogensiloxane (c1) and of a hydrosilylation reaction catalyst (c2).

Particularly preferred organohydrido silicon compounds (c1) are polymers or copolymers with RHSiO units ended with either R″₃SiO_{1/2} or HR″₂SiO_{1/2}, wherein R″ is independently selected from alkyl radicals having 1 to 20 carbon atoms, phenyl or trifluoropropyl, preferably methyl. It is also preferred that the viscosity of component (C) is about 0.5 to 1,000 MPa-s at 25° C., preferably 2 to 500 MPa·s. Further, this component preferably has 0.5 to 1.7 weight percent hydrogen bonded to silicon. It is highly preferred that component (c1) is selected from a polymer consisting essentially of methylhydridosiloxane units or a copolymer consisting essentially of dimethylsiloxane units and methylhydridosiloxane units, having 0.5 to 1.7 percent hydrogen bonded to silicon and having a viscosity of 2 to 500 MPa-s at 25 C. It is understood that such a highly preferred system will have terminal groups selected from trimethylsiloxy or dimethylhdridosiloxy groups.

Component (c1) may also be a combination of two or more of the above described systems. The organohydrido silicon compound (C) is used a level such that the molar ratio of SiH therein to Si-alkenyl in component (B) is greater than 1 and preferably below about 50, more preferably 3 to 20, most preferably 6 to 12.

These SiH-functional materials are well known in the art and many of them are commercially available.

### LSR or HCR with Fillers

LSR or HCR with fillers can be used as component (B). Any suitable filler or combination of fillers may be utilised. The elastomeric composition may contain one or more finely divided, reinforcing fillers, such as fumed or precipitated silica, and/or calcium carbonate, and/or non-reinforcing fillers, such as crushed quartz, diatomaceous earths, barium sulfate, iron oxide, titanium dioxide, carbon black, talc, and wollastonite. Other fillers which might be used alone or in addition to the above include aluminite, calcium sulfate (anhydrite), gypsum, calcium sulfate, magnesium carbonate, clays, e.g. kaolin, aluminium trihydroxide, magnesium hydroxide (brucite), graphite, copper carbonate, e.g. malachite, nickel carbonate, e.g. zarachite, barium carbonate, e.g. witherite and/or strontium carbonate e.g. strontianite, aluminium oxide, silicates selected from olivine group, garnet group, aluminosilicates, ring silicates, chain silicates and sheet silicates (the olivine group comprises silicate minerals, such as but not limited to, forsterite and Mg₂SiO₄; the garnet group comprises ground silicate minerals, such as but not limited to, pyrope, Mg₃Al₂Si₃O₁₂, grossular, and Ca₂Al₂Si₃O₁₂; aluninosilicates comprise ground silicate minerals, such as but not limited to, sillimanite, Al₂SiO₅, mullite, 3Al₂O₃.2SiO₂, kyanite, and Al₂SiO₅; the ring silicates group comprises silicate minerals, such as but not limited to, cordierite and Al₃(Mg,Fe)₂[Si₄AlO₁₈]; the chain silicates group comprises ground silicate minerals, such as but not limited to, wollastonite and Ca[SiO₃]; the sheet silicates group comprises silicate minerals, such as but not limited to, mica, K₂Al₁₄[Si₆Al₂O₂₀](OH)₄, pyrophyllite, Al₄[Si₈O₂₀](OH)₄, talc, Mg₆[Si₈O₂₀](OH)₄, serpentine for example, asbestos, Kaolinite, Al₄[Si₄O₁₀](OH)₈, and vermiculite) and silicone resins.

### Description of Filler Treatment

Fillers may be surface treated. A surface treatment of the filler(s) may be performed, for example with a fatty acid or a fatty acid ester such as a stearate, or with organosilanes, organosiloxanes, or organosilazanes hexaalkyl disilazane or short chain siloxane diols to render the filler(s) hydrophobic and therefore easier to handle and obtain a homogeneous mixture with the other components The surface treatment of the fillers makes them more easily wetted by the silicone polymer. These surface-modified fillers do not clump, and can be homogeneously incorporated into the silicone polymer. Furthermore, the surface-treated fillers give a lower conductivity than untreated or raw material.

Silanes found to be most suitable for the treatment of the fillers are alkoxysilanes of the general formula R²₍₄₋ₙ₎Si(OR²)ₙ, wherein n has a value of 1-3; and each R² is the same or different and represents a monovalent organic radical such as an alkyl group, an aryl group, or a functional group such as an alkenyl group, e.g. vinyl or allyl, an amino group or an amido group. Some suitable silanes therefore include alkyltrialkoxysilanes such as methyltriethoxysilane, methyltrimethoxysilane, phenyl tialkoxysilanes such as phenyltrimethoxysilane, or alkenyltrialkoxysilanes such as vinyltriethoxysilane, and vinyltrimethoxysilane. If desired, silazanes can also be used as treating agents for the mixture of aluminium trihydroxide and kaolin filler. These include, but are not restricted to, hexamethyldisilazane, 1,1,3,3-tetramethyldisilazane and 1,3-divinyltetramethyldisilazane. Short chain polydiorganosiloxanes might for example include hydroxy terminated polydimethylsiloxanes having a degree of polymerisation of from 2 to 20, hydroxy terminated polydialkyl alkylalkenylsiloxanes having a degree of polymerisation of from 2 to 20.

The proportion of such fillers when employed will depend on the properties desired in the elastomer-forming composition and the elastomer. Usually the filler content of the composition will reside within the range 5-500 parts by weight per 100 parts by weight of the polymer.

### Other Ingredients

Other ingredients which may be included in the compositions of the material include but are not restricted to co-catalysts for accelerating the cure of the composition such as metal salts of carboxylic acids and amines, rheological modifiers, adhesion promoters, pigments, colouring agents, desiccants, heat stabilizers, flame retardants, UV stabilisers, chain extenders, cure modifiers, electrically and/or heat-conductive fillers, blowing agents, anti-adhesive agents, handling agents, peroxide cure co-agents, acid acceptors, fungicides and/or biocides and the like (which may suitably by present in an amount of from 0 to 0.3% by weight), water scavengers, (typically the same compounds as those used as cross-linkers or silazanes). It will be appreciated that some of the additives are included in more than one list of additives. Such additives would then have the ability to function in each manner as stated.

Heat stabilizers may include antioxidants, UV absorbers, HALS for the main.

Flame retardants may include for example, carbon black, hydrated aluminium hydroxide, hydrated magnesium hydroxide and silicates such as wollastonite, and carbonates.

Electrically conductive fillers may include carbon black, metal particles such as silver particles any suitable, electrically conductive metal oxide fillers such as titanium oxide powder whose surface has been treated with tin and/or antimony, potassium titanate powder whose surface has been treated with tin and/or antimony, tin oxide whose surface has been treated with antimony, and zinc oxide whose surface has been treated with aluminium.

Thermally conductive fillers may include metal particles such as powders, flakes and colloidal silver, copper, nickel, platinum, gold aluminium and titanium, metal oxides, particularly aluminium oxide (Al₂O₃) and beryllium oxide (BeO), magnesium oxide, zinc oxide, zirconium oxide, ceramic fillers such as tungsten monocarbide, silicon carbide and aluminium nitride, boron nitride and diamond.

Optional diluents to be used with HCRs (and possibly with LSRs of higher viscosities) include aliphatics, namely white spirit, Stoddard solvent, hexane, heptane, c-hexane, and aromatics such as toluene and xylene.

### Description of a Non Cross-Linked and Non Reactive Silicone Fluid

Component (B) can be a non cross-linked and substantially non reactive silicone fluid. The non cross-linked and non reactive silicone fluid may be a polydiorganosiloxane described by the following formula:

R³₃SiO[(R³₂SiO)ₙ]SiR³₃

wherein each R³ is the same or different and represents C₁₋₁₈ alkyl (preferably C₁₋₈ alkyl and more preferably C₁₋₄ alkyl) or aryl (e.g. phenyl or naphthyl), either of which may optionally be further substituted with non-reactive groups, such as fluoro (e.g. a trifluoroalkyl group); preferably each R³ group is a methyl or ethyl group. It is typically a trialkyl silyl terminated polydimethylsiloxane (PDMS) fluid. Most preferably each terminal alkyl group is either methyl or ethyl but are not necessarily the same.

Alternatively, or in addition, the non-reactive silicone fluid may contain a polyorganosiloxane having a degree of branching due to the presence of one or more of either or both of the following groups in the R³₃SiO[(R³₂SiO)ₙ]SiR³₃ polymer backbone:

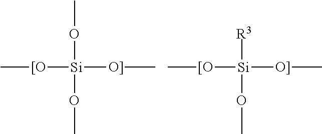

wherein R³ is as described hereinabove.

The value of n is such that the Brookfield viscosity of the polymer is 2,000-3,000,000 mPas, preferably 5,000-1,000,000 mPas, more preferably 10,000-500,000 mPas, at 25° C.

The pressure material can be obtained from thermoplastic pellets obtained by processing a mixture (A)+(B) or (A)+(a)+(b)+(c) described above into an extruder. In another aspect, the material is directly obtained in the form of a sheet or a moulded article with no pellet manufacturing step. The different components or precursors of the material may be moulded into any moulded shapes. For instance, the components or precursors of the material may be moulded into a sheet material using conventional one screw extruder and an appropriate die. Preferably, the sheet material has a thickness of 1-30 mm. The sheet material may be formed by reinforcing the above described composition of the material with fibres. Carbon, polyester, polyimide, polyaramide, polyolefin, polyimide, polyacrylonitrile, polyethylene tyerephtalate (PTFE), cotton, glass, silica fibers can be used.

The material used for impact protection, in the form of a sheet or a moulded article, can be foamed or unfoamed. In particular, the material can be foamed in closed cell foam as described in WO 2005/000966. When the composition is converted to a foamed sheet or foamed moulded article, it is possible to use any chemical or mechanical blowing agent used with thermoplastic materials. The sheet or article can be foamed with an addition of expandable hollow or plastic microspheres to the composition, during the conversion to the foamed sheet or foamed moulded article. According to another aspect, the foamed sheet or foamed moulded article can be fully cross-linked by a beam or by peroxides and suitable co agents added to the composition.

The material in the form of a sheet can be laminated to other sheets of the material or one or more alternative substrates, for instance by calandering or by the use of adhesives and/or welding techniques. Examples of such substrates are fabrics or non-woven materials. In particular, it may also be associated with a textile layer or similar where the textile has the facility to enhance the abrasion performance and in some cases the resistance to intrusion from sharp objects and/or assist in the attachment of the composite material to other systems or products. A stretchable textile backing will also serve to limit the elongation of the material and thereby provide durability. The textile may also serve as an antiballistic or stab-proof fabric such as certain woven grades of KEVLAR.

According to another embodiment of the invention, the material can be used in the form of a laminate obtained by coextruded layers of thermoplastic materials including the material used according to the invention.

The sheet or laminate may also be in the form of a shaped article, for example so that it conforms to the contours of the human or animal body, e.g. to form shaped material. It may also be formed into a garment. This may be achieved by, for example, thermoforming or overmoulding sheets. Sheets and/or laminates may additionally be embossed or otherwise marked, if required.

The composition may also be moulded into any mouldable shapes by injection moulding or compression moulding.

According to another embodiment of the invention, the material can be in the form of fibers. The fibers may be woven, knitted or otherwise configured such as to incorporate air into the final article. When such a material is subjected to impact, the distortion of each fiber is facilitated by the air spaces to provide a large number of localized bending deflections, which is preferable for the efficient use of the composite material in absorbing impact.

According to another embodiment, the pressure material is a textile material as defined hereinbefore. Such materials are defined in our WO2010/072881 A1 published on 1 Jul. 2010, and incorporated herein by way of reference.

### Description of Component (c)

In this regard Component (c) is an elastomeric material having a modulus at 100% elongation of 0.1-10 MPa. Preferably the modulus at 100% elongation is 0.5-9 MPa. The modulus at 100% elongation may be determined by the process described in ASTM D638-97. However, some elastomers cannot be elongated to 100% and in such cases the modulus may be determined at lower elongation values and extrapolated to 100%.

The elastomeric material may be a natural elastomer, such as a latex rubber, or a synthetic elastomer. Examples of suitable synthetic elastomers include neoprene; polyester; polyurethane, such as Witcoflex 959 Matt from Baxenden Chemicals Ltd which is a solvent-based single component polyurethane solution in isopropanol and toluene and has a modulus at 100% elongation of 3.5 MPa; ethylene/vinyl acetate copolymer (EVA); EP rubbers such as EPDM rubbers; or copolymers including those having an olefin block, such as polypropylene or an ethylene in conjunction with softer blocks. Such elastomeric materials can be provided a polymers in bead form which may be melt-processable, provided in solution or provided in emulsion, or they may be provided a precursors which are then reacted with other ingredients, for example where the material is a polyurethane precursor, they may be reacted with isocyanates. These elastomeric materials may also be cross-linked, for example polyurethanes cross-linked using hydroxy and/or amine terminated cross-linking agents. Preferably the elastomeric materials are a non-thermoplastic elastomer.

More preferably, however, the elastomeric material is a siloxane material, in which case the elastomer is formed by cross-linking a siloxane polymer material, or polyorganosiloxane. Preferably borated silicone polymers are excluded from component (c), in particular those exhibiting dilatant properties. The polymer and the cross-linking conditions are not critical, provided that the cured cross-linked elastomer formed has the required modulus at 100% elongation. Examples of the cross-linking (curing) reactions include: cross-linking a polyorganosiloxane having alkenyl or alkynyl functional groups and a polyorganohydrogensiloxane in the presence of a hydrosilylation catalyst (a platinum-type catalyst) and cross-linking α,ω-dihydroxypolydiorganosiloxane with a hydrolysable group-containing organosilane in the presence of a condensation catalyst. Other cure systems such as cross-linking a polyorganosiloxane having alkenyl or alkynyl functional groups in the presence of an organic peroxide catalyst may be used but are not preferred. This is because peroxide type catalysts function via a free-radical reaction pathway (i.e. free radical initiated) and may potentially lead to the cross-linking of components (c) and (d) of the composition described herein.

In one embodiment, the elastomer is obtained from a curable silicone elastomer-forming composition based on a polyorganosiloxane having alkenyl or alkynyl functional groups and a polyorganohydrogensiloxane in the presence of a hydrosilylation catalyst (a platinum-type catalyst). Such curable silicone elastomer-forming composition comprises (i) an organopolysiloxane polymer having at least two alkenyl or alkynyl groups per molecule, preferably at least one of which is and most preferably at least two of which are end group(s) and optionally alkenyl or alkynyl groups linked to silicon atoms along the polymer backbone, preferably (ii) a filler, typically treated with a hydrophobing agent, and (iii) a cure package having a siloxane cross-linker containing at least three Si—H groups per molecule and a hydrosilylation catalyst.

In the case of the curable silicone elastomer-forming composition being a liquid silicone rubber (LSR) composition as defined hereinabove.

The curable silicone elastomer-forming composition may also be a diluted high consistency rubber (HCR) as defined hereinabove.

Any suitable filler or combination of fillers may optionally be utilised as described hereinabove.

Alternatively, the filler may comprise an organopolysiloxane resin. The organopolysiloxane resin may be exemplified by resins comprising: the (CH₃)₃SiO_{1/2} unit and SiO_{4/2} unit; the (CH₃)₃SiO_{1/2} unit, (CH₂═CH)SiO_{3/2} unit, and SiO_{4/2} unit; the (CH₂═CH)(CH₃)₂SiO_{1/2} unit and SiO_{4/2} unit; and the (CH₂═CH)(CH₃)₂SiO_{1/2} unit, (CH₂═CH)SiO_{3/2} unit, and SiO_{4/2} unit. Among these resins, the vinyl-containing resins are preferred because they lead to an improvement in the strength of the silicone rubber coating membrane.

In another embodiment, component (c) is based on the cross-linking of a polyorganosiloxane preferably having one or more alkenyl or alkynyl functional groups in the presence of an organic peroxide. As previously indicated this is not a preferred route as the use of this free radical initiated cure system is potentially likely to lead to cure of component (d) in with component A however, if used polyorganosiloxane having alkenyl or alkynyl functional groups is as described hereinabove is preferred. The curing agent is an organic peroxide, such as dialkyl peroxide, diphenyl peroxide, benzoyl peroxide, 1,4-dichlorobenzoyl peroxide, paramethyl benzoyl peroxide, 2,4-dichlorobenzoyl peroxide, di-t-butyl peroxide, dicumyl peroxide, tertiary butyl-perbenzoate, monochlorobenzoyl peroxide, ditertiary-butyl peroxide, 2,5-bis-(tertiarybutyl-peroxy)-2,5-dimethylhexane, 1,1-bis(t-butylperoxy)-3,3,5-trimethylcyclohexane tertiary-butyl-trimethyl peroxide, tertiary-butyl-tertiary-butyl-tertiary-triphenyl peroxide, and t-butyl perbenzoate. The most suitable peroxide based curing agents are benzoyl peroxide, 2,4-dichlorobenzoyl peroxide, di-t-butyl peroxide, and dicumyl peroxide. Organic peroxides such as the above are particularly utilised when R¹ in the polymer as defined hereinabove is an alkyl group but the presence of some unsaturated hydrocarbon groups per molecule is preferred. Clearly, in this embodiment, the polyorganohydrogensiloxane and hydrosilylation catalyst are not required, although the filler is still preferably used to achieve the necessary 100% modulus.

In a further embodiment, the elastomer may be obtained by cross-linking α,ω-dihydroxypolydiorganosiloxane or a polydiorganosiloxane with two or more hydrolysable groups with a suitable hydrolysable crosslinker having at least 3 hydrolysable groups such as for example an organosilane. The polymer backbone is essentially the same as that described hereinabove, but with optional hydrolysable groups rather than reactive unsaturated groups. However, the polymer end groups are different.

Any suitable hydrolysable cross-linker may be used with the above. The cross-linker may be a silane compound containing at least 3 hydrolysable groups. These include one or more silanes or siloxanes which contain silicon-bonded hydrolysable groups such as acyloxy groups (for example, acetoxy, octanoyloxy, and benzoyloxy groups); ketoximino groups (for example dimethyl ketoximo, and isobutylketoximino); alkoxy groups (for example methoxy, ethoxy, and propoxy) and alkenyloxy groups (for example isopropenyloxy and 1-ethyl-2-methylvinyloxy).

In the case of siloxane based cross-linkers the molecular structure can be straight-chained, branched or cyclic.

Some of the cross-linker may have two condensable groups but the majority preferably have three or four silicon-bonded condensable (preferably hydroxyl and/or hydrolysable) groups per molecule which are reactive with the condensable groups in the organopolysiloxane polymer. When the cross-linker is a silane and when the silane has three silicon-bonded hydrolysable groups per molecule, the fourth group is suitably a non-hydrolysable silicon-bonded organic group. These silicon-bonded organic groups are suitably hydrocarbyl groups which are optionally substituted by halogen such as fluorine and chlorine. Examples of such fourth groups include alkyl groups (for example methyl, ethyl, propyl, and butyl); cycloalkyl groups (for example cyclopentyl and cyclohexyl); alkenyl groups (for example vinyl and allyl); aryl groups (for example phenyl, and tolyl); aralkyl groups (for example 2-phenylethyl) and groups obtained by replacing all or part of the hydrogen in the preceding organic groups with halogen. Preferably however, the fourth silicon-bonded organic groups is methyl.

Silanes and siloxanes which can be used as cross-linkers include alkyltrialkoxysilanes such as methyltrimethoxysilane (MTM) and methyltriethoxysilane, alkenyltrialkoxy silanes such as vinyltrimethoxysilane and vinyltriethoxysilane, isobutyltrimethoxysilane (iBTM). Other suitable silanes include ethyltrimethoxysilane, vinyltriethoxysilane, phenyltrimethoxysilane, alkoxytrioximosilane, alkenyltrioximosilane, 3,3,3-trifluoropropyltrimethoxysilane, methyltriacetoxysilane, vinyltriacetoxysilane, ethyl triacetoxysilane, di-butoxy diacetoxysilane, phenyl-tripropionoxysilane, methyltris(methylethylketoximo)silane, vinyl-tris-methylethylketoximo)silane, methyltris(methylethylketoximino)silane, methyltris(isopropenoxy)silane, vinyltris(isopropenoxy)silane, ethylpolysilicate, n-propylorthosilicate, ethylorthosilicate, dimethyltetraacetoxydisiloxane.

The cross-linker used may also comprise any combination of two or more of the above.

The composition of this further embodiment may further comprises a condensation catalyst. This increases the speed at which the composition cures.

For the avoidance of doubt an unbranched secondary alkyl group is intended to mean a linear organic chain which does not have a subordinate chain containing one or more carbon atoms, i.e. an isopropyl group, whilst a branched secondary alkyl group has a subordinate chain of one or more carbon atoms such as 2,4-dimethyl-3-pentyl.

Certain additional components may optionally be included in the elastomer-forming composition to be used in the present invention. To obtain a longer working time or “pot life”, the activity of hydrosilylation catalysts under ambient conditions can be retarded or suppressed by addition of a suitable inhibitor. Known platinum-group metal catalyst inhibitors include the acetylenic compounds disclosed in U.S. Pat. No. 3,445,420. Acetylenic alcohols such as 2-methyl-3-butyn-2-ol and 1-ethynyl-2-cyclohexanol constitute a preferred class of inhibitors that suppress the activity of a platinum-based catalyst at 25° C. Compositions containing these catalysts typically require heating at temperatures of 70° C. or above to cure at a practical rate. Room temperature cure is typically accomplished with such systems by use of a two-part system in which the cross-linker and inhibitor are in one of the two parts and the platinum is in the other part. The amount of platinum is increased to allow for curing at room temperature.

Inhibitor concentrations as low as one mole of inhibitor per mole of platinum-group metal will, in some instances, impart satisfactory storage stability and cure rate. In other instances inhibitor concentrations of 500 or more moles of inhibitor per mole of platinum-group metal are required. The optimum concentration for a given inhibitor in a given composition can readily be determined by routine experimentation.

Additional components can be added to the hydrosilylation composition which are known to enhance such reactions. These components include salts such as sodium acetate which have a buffering effect in combination with platinum-type catalysts.

Other ingredients which may be included in the compositions include but are not restricted to co-catalysts for accelerating the cure of the composition such as metal salts of carboxylic acids and amines, rheological modifiers, adhesion promoters, pigments, colouring agents, desiccants, heat stabilizers, flame retardants, UV stabilisers, chain extenders, cure modifiers, electrically and/or heat-conductive fillers, blowing agents, foaming agents, anti-adhesive agents, handling agents, peroxide cure co-agents, acid acceptors, fungicides and/or biocides and the like (which may suitably by present in an amount of from 0 to 0.3% by weight), water scavengers, (typically the same compounds as those used as cross-linkers or silazanes). It will be appreciated that some of the additives are included in more than one list of additives. Such additives would then have the ability to function in each manner as stated.

The rheological additives include silicone organic co-polymers such as those described in EP 0 802 233 based on polyols of polyethers or polyesters, non-ionic surfactants selected from the group consisting of polyethylene glycol, polypropylene glycol, ethoxylated castor oil, oleic acid ethoxylate, alkylphenol ethoxylates, copolymers or ethylene oxide (EO) and propylene oxide (PO), and silicone polyether copolymers; as well as silicone glycols.

Adhesion promoter(s) may also be incorporated. These may include alkoxysilanes such as aminoalkylalkoxy silanes, epoxyalkylalkoxy silanes, for example, 3-glycidoxypropyltrimethoxysilane and mercapto-alkylalkoxy silanes and γ-aminopropyl triethoxysilane, reaction products of ethylenediamine with silylacrylates. Isocyanurates containing silicon groups such as 1,3,5-tris(trialkoxysilylalkyl) isocyanurates may additionally be used. Further suitable adhesion promoters are chelated materials or reaction products of epoxyalkylalkoxy silanes such as 3-glycidoxypropyltrimethoxysilane with amino-substituted alkoxysilanes such as 3-aminopropyltrimethoxysilane and optionally alkylalkoxy silanes such as methyl-trimethoxysilane, epoxyalkylalkoxy silane, mercaptoalkylalkoxy silane, and derivatives thereof.

Handling agents are used to modify the uncured properties of the silicone rubber such as green strength or processability sold under a variety of trade names such as SILASTIC® HA-1, HA-2 and HA-3 sold by Dow Corning Corporation).

Peroxide cure co-agents are used to modify the properties, such as tensile strength, elongation, hardness, compression set, rebound, adhesion and dynamic flex, of the cured rubber. These may include di- or tri-functional acrylates such as trimethylolpropane triacrylate and ethylene glycol dimethacrylate; triallyl isocyanurate, triallyl cyanurate, polybutadiene oligomers and the like. Silyl-hydride functional siloxanes may also be used as co-agents to modify the peroxide catalysed cure of siloxane rubbers.

The acid acceptors may include magnesium oxide, calcium carbonate, zinc oxide and the like.

The ceramifying agents can also be called ash stabilisers and include silicates such as wollastonite.

Plasticisers or extenders may be utilised, if required. Examples include those reviewed in GB 2 424 898, and those described in WO 2008/045417 but in view of component (d) typically no organic type plasticiser and/or extender (sometimes referred to as a processing aid) will be additionally required.

### Description of Component (d)

Component (d) is a substantially non-reactive silicone fluid. Preferably Component (d) is totally non-reactive, although small amounts of reactivity can be tolerated in most instances. By non-reactive is meant that it does not react chemically (to form a covalent bond) with the pre-cursors of component (c). The non-reactive silicone fluid may be a polyorganosiloxane as described and defined hereinabove

The (ii) composition of the present invention is obtained by forming an intimate mixture of components (c) and (d). In one embodiment, the composition consists essentially of components (c) and (d). By essentially, is meant that conventional additives such as those described hereinabove, but which do not adversely affect the energy-absorption properties, may be included. The composition preferably contains 5-80% by weight of component (d), based on the totally weight of the composition, and preferably 20-60% by weight, and most preferably 30-50%. The resulting composition exhibits viscoelastic properties. As used herein, the term viscoelastic refers to the property of exhibiting shear-rate dependent strain, having both liquid (linear strain when stressed) and elastic (instantaneous strain when stressed) properties. For the avoidance of doubt, this is a different property to dilatancy which means that under impact conditions, the strain rate is very high and the composition exhibits elastic properties, whereas under normal conditions when strained slowly, the composition exhibits substantially viscous properties.

Alternative ways of improving the compatibility of components (c) and (d) or of the precursors of component (c) with component (d), in order to ensure the intimate mixture could include the use of emulsions, solvents or other dispersing aids.

The composition described in the use of the present invention is preferably prepared by forming component (c) in the presence of component (d) and is thus formed by intimately mixing the curable elastomer-forming composition which may cure into component (c) together with component (d). It therefore preferably comprises the steps of (a) mixing (i) a curable polymer, e.g. a polyurethane precursor or an polyorganosiloxane, (ii) potentially a filler, (iii) a curing package, e.g. isocyanate or a silicone based crosslinker and where necessary a catalyst and (c) a non-reactive silicone fluid, having a viscosity of 1,000-3,000,000 mPas at 25° C.; and (b) curing the resultant mixture. Typically all the ingredients are individually pre-prepared and introduced individually into the mixture. The components (i)-(iii) and (c) are as described hereinabove. Preferably curing is performed by heating the resultant mixture. It is possible to perform the curing in stages and it can be envisaged to mix part of components (c) and (d), followed by some curing, followed by further mixing and curing of remaining parts of the relevant components.

Thus, component (c) is formed by reacting or cross-linking its component parts in the presence of component (d) allowing component (d) to be dispersed throughout the matrix formed by component (c). However, component (d) is substantially unreactive and hence does not become covalently bonded to component (c) to any large extent. By unreactive (or non-reactive) is meant that component (d) does not react with the precursors of component (c) during the cross-linking (curing) process and hence does not participate in the cross-linking (or chain extension) reaction. Clearly, the chemical nature of component (d) will depend on the nature of the curing reaction. For example, a vinyl-substituted polyorganosilane would be considered reactive if component (c) is prepared by cross-linking using the hydrosilylation reaction or siloxanes, but would not be reactive if component (c) is prepared by a condensation reaction or siloxanes. This forms an intimate mixture of the two components, i.e. a mixture in which component (d) is dispersed throughout component (c). It is a mixture as the two components are not chemically bonded (covalently bonded) to one another. Although not wishing to be bound by theory, it is believed that the intimate mixture of these two components, together with the high viscosity of component (d), allows component (d) to resist flow under impact.

The composition may be formed into a pressure material. The pressure material may be formed solely of the composition of the present invention, particularly where the composition is self-supporting. For example, the pressure material may be composed of a foam based on a matrix formed from the composition of the present invention. The composition is treated with a foaming agent during the curing process so that sheet material is in the form of a foam composed solely of the elastomeric material, which could be for example a polyurethane or a silicone composition, having component (d) distributed throughout the elastomeric foam. The pressure material may also comprise the composition of the present invention together with a reinforcing material, such as reinforcing fibres, e.g. polyester, polyamide, polyaramide, polyolefin, polyimide, polyacrylonitrile, PTFE, cotton, carbon fibres, glass fibre and/or silica fibres.

Alternatively, the pressure material may be formed of a substrate together with the elastomer composition of the present invention. The substrate supports the composition and provides structural integrity (indeed, the substrate would typically have structural integrity in the absence of the composition). The substrate may be impregnated and/or coated with the elastomer composition. Where the substrate is impregnated, the substrate has voids/cavities into which the composition may enter. Although any amount of the composition will improve the performance of a substrate compared to the untreated substrate, preferably the composition is present at 100-5,000 g/m², more preferably 500-3,000 g/m².

The substrate may be a fabric, such as a woven fabric (e.g. a fleece material), a non-woven fabric or a knitted fabric (often in the form of and/or sold as a spacer textile, typically a 3 dimensional spacer textile). The fabric may be formed of any suitable material, such as, for example, polyester, polyamide, polyolefin, aromatic polyamide, cotton, wool, acrylic or cellulosic fibres. It may be constructed with an abrasion resistant fibre such as aromatic polyamide arranged to be at the outer surface of a protective garment with a comfort fibre, such as cotton or a wicking microfiber, at the inner surface.

The substrate may be impregnated with the composition of the present invention. The composition may need to be diluted with an organic solvent to the optimum viscosity for application to the substrate. Examples of suitable solvents are aliphatics, namely white spirit, Stoddard solvent, hexane, heptane, c-hexane, and aromatics such as toluene and xylene. The solvent can be a supercritical fluid, for example supercritical carbon dioxide. The concentration of composition in such a solution may, for example, be 10-95% by weight, usually from 20-80% by weight. The composition may also be introduced into or onto the substrate by providing the composition in emulsion form.

After impregnation the sheet material is dried, either by allowing the fabric to dry under ambient conditions or by applying heat and/or a current of a drying gas such as air to accelerate drying. Drying can for example be carried out at 40-200° C., particularly 80-180° C.

The substrate may also be a foam, such as an open cell, partially open cell, or closed cell foam, e.g. a polyurethane foam or cellulose foam or foam materials made from individual foamed beads linked together by melt bonding or chemical binder materials.

A foam impregnated with a composition can be produced by mixing the composition with foam-forming ingredients which are then allowed to foam. The foam-forming ingredients may be a plastics material mixed with a latent-gas-generating material but are preferably reagents which react to form a foam blown with gas generated during the reaction, for example polyurethane foam precursors such as an isocyanate or blocked isocyanate and an active hydrogen compound such as a polyol, particularly a polyether polyol and/or a polyester polyol.

The composition of the present invention may be mixed with the foam-forming ingredients prior to or after forming and curing the foam.

According to one embodiment of the invention, the substrate is an auxetic material, that is a material having a negative or effectively negative Poisson ratio so that it expands perpendicular to an axis about which it is stretched. Auxetic materials are described, for example, in WO 2004/088015, WO 00/53830, U.S. Pat. No. 4,668,557 and WO 91/01210. The pressure protection can be enhanced by using an auxetic material as the substrate.

The pressure material of the present invention may include a substrate which is a resilient carrier with voids or cavities therein, as described in WO 03/022085, but employing the composition of the present invention rather than the dilatant material described therein. The resilient carrier is coated, impregnated or combined with the composition of the present invention such that the resilient carrier supports the composition.

The following preferred embodiments for the resilient carrier described in WO 03/022085 apply equally to the substrate of the present invention. Thus, the substrate may be a spacer material; the spacer material may comprise a resilient core sandwiched between a pair of covering layers. This may take the form of a ribbed material sandwiched between a top sheet and a bottom sheet (see FIGS. 1 and 2 and the accompanying text of WO 03/022085) or resilient partitions which are sandwiched between and joined to a top sheet and a bottom sheet (see FIGS. 5 and 6 and the accompanying text). Of course, it is also possible to provide only one sheet on which the elastomer material is provided. An alternative is a “hex-type” spacer material (see FIG. 7 and the accompanying text of WO 03/022085). The outer surface of each covering layer may be formed with a plurality of compressible bubbles therein (see FIG. 8 and the accompanying text of WO 03/022085), and elongate hollow channels may be formed in the compressible core. In addition, upper and lower textile layers may be formed with a plurality of pockets formed therein by stitching, with the pockets filled with the composition of the present invention, e.g. impregnated in the fibres (see FIG. 9 and the accompanying text). One advantage of using such materials is their breathability when composition described in the present invention is applied to the substrate but does not fill the hollow channels or apertures therein.

The substrate based on that disclosed in WO 03/022085 may also have holes formed there through. The substrate may also be a foam (see FIGS. 3 and 4 and the accompanying text). Alternatively, the pressure material may be formed of discrete modules made of composition of the present invention sandwiched between a pair of covering layers (see FIGS. 10-13 and the accompanying text). The modules may be randomly arranged in the compressible core, arranged in axially aligned rows across the width of the sheet or as parallel elongate hollow tubular members. Also, each module may have a covering layer thereon. The modules may be spherical and they may be hollow or have a lightweight centre. The pressure material may also be formed into a shaped article, e.g. a knee or elbow pad, or a shoe (see FIGS. 21-23 and 25 and the accompanying text).

The pressure material of the present invention may alternatively include a substrate as disclosed in WO 03/055339. This embodiment of the present invention, based on WO 03/055339, provides a self-supporting energy absorbing composite comprising a solid foamed synthetic polymer matrix and the composition of the present invention. The matrix is preferably elastic, more preferably a synthetic elastomer, and most preferably an elastomeric polyurethane.

In a preferred embodiment, the self-supporting energy absorbing composite is a foam and the composition of the present invention is contained in the pores of the foam.

The foam may be an open-cell, closed-cell or part-open-part-closed foam. The foam recovers after being subjected to compression and recovery is preferably complete after 5 seconds or less and more preferably 2 seconds or less. The composition of the present invention is preferably included during the formation of the foam.

An example of the base polyurethane system is that available as J-Foam 7087 from Jacobson Chemicals Ltd in Farnham, Surrey. Further details are given in Examples 1 and 2 of WO 03/055339.

Alternatively, rather than being a substrate for the composition of the present invention, the composition of the present invention may be formed into an pressure material without a substrate, as described hereinabove, but in the form of the material described in WO 03/055339. That is, component (c) of the present invention may be the solid foamed synthetic polymer matrix of WO 03/055339 and component (d) of the present invention may take the place of the dilatant of WO 03/055339. However, the composition of the present invention (by using an elastomer of the specified modulus, and a fluid with the specified viscosity range) provides improved impact resistance.

WO 03/055339 also provides a cross-reference to JP 06-220242. JP 06-220242 discloses an impact cushioning material obtained by coating the surface of a skeletal lattice of a flexible three-dimensional network or foam having continuous internal voids with a silicone bouncing putty. The pressure material of the present invention may also be based on this skeletal lattice as the substrate. This lattice may be exemplified by plastic foams that have an open cell structure, for example, polyethylene, polystyrene, polyvinyl chloride, polyurethane, phenolic resin, urea resin, methacrylic resin, or silicone resin; by porous natural materials, e.g. sponge and cork; or by porous materials composed of a fibrous substance, e.g. woven fabrics and nonwoven fabrics.

The pressure material of the present invention is preferably in the form of a sheet, e.g. with a thickness of 1-30 mm. The sheet may have a uniform thickness or the thickness may vary within the range of 1-30 mm. The sheet may also be composed of a plurality of layers which together form the sheet having the desired thickness.

The composition described in the present invention may be applied by any suitable method of application. Examples include but are not restricted to spray coating, curtain coating, die coating, dip coating, knife coating and screen coating.

The item of wear incorporating the pressure material described in the present invention may be for contact sports, high risk sports and activities or the like such as, but not restricted to, rugby, soccer, American football, baseball, basketball, martial arts, boxing, sailing, windsurfing, wakeboarding, ice-skating, speedskating, snowboarding, skiing, ice-hockey, field hockey, roller hockey, roller blading, cricket, hurling, lacrosse, mountain biking, cycling, bobsleigh, extreme sports e.g. bungee jumping weightlifting and motorcycling.

The pressure material may also be used in medical applications e.g. for hip protection, head protection for vulnerable people, protective devises to aid recovery from injury and/or orthopaedic devices or in work protection wear e.g. safety gloves, safety footwear, safety clothing.

Another application for the pressure material is in the protection of items or articles into which the pressure material may be incorporated or which may use the pressure material as an encasement e.g. suitcases, laptop cases, laptop backpacks, camera cases, mobile phone cases, portable music equipment cases, golf clubs, surfboard protection, radio and in packaging for fragile items in transportation, lining of vehicles and crates for transportation. Furthermore, the pressure material may be used in transportation applications such as automobile dashboards, bumpers, and safety equipment in other transport e.g. trains and aeroplanes.

In use a plurality of layers of the treated impact protection material may be utilised in order to suit the application for which it is to be used. The layers may be identical or may be a combination of alternative substrates described above or alternatively may be a combination one or more layers of impact protection material according to the present invention and layers of other materials. Furthermore, the composition described in the present invention may be applied alone to a substrate or may be applied with other suitable materials which do not negatively affect the impact resistance of the treated materials. Examples might include gels, resins and foams or the like.

Unless otherwise indicated all viscosity values provided are in mPa·s and were measured at 25° C. and are Brookfield viscosity. 1 mPa·s is 0.1 Poise A poise is a cgs unit of viscosity equal to the tangential force in dynes per cm² required to maintain a difference in velocity of 1 cms⁻¹ between 2 parallel planes of a fluid separated by 1 cm. This is measured using a rotational flow method, which uses a rotating spindle immersed in the test liquid and measures torque and hence resistance to flow of the liquid. Typically measurements are taken using a Brookfield rotational viscometer, Model DVIII with a spindle CP52 at 0.5 rpm, measured at 25° C.

The pressure material may also be in the form of a shaped article, for example so that it conforms to the contours of the human or animal body, e.g. knee, elbow or shoulder protection. Examples e.g. for preventing/protecting the wearer from blunt trauma include (in each case as a separate protector or formed into a garment or included as part of a garment, elbow protectors, knee protectors, forearm protectors, thigh protectors, chest protectors, back protectors, shoulder, lower leg protectors and chest protectors shinguards, shin protectors, helmets, head protectors, hip protectors, gloves, kidney protection and coccyx protection. The pressure material may also be in the form of footwear—e.g. heel of the shoe, forefoot, shoe upper or may be in protective sports equipment—e.g. rugby post protectors, training equipment, landing mats, cricket pads and gloves etc.

The protective equipment incorporating the pressure material described in the present invention may be for contact sports, high risk sports and activities or the like such as but not restricted to rugby, soccer, American football, baseball, basketball, martial arts, boxing, sailing, windsurfing, wakeboarding, ice-skating, speedskating, snowboarding, skiing, ice-hockey, field hockey, roller hockey, roller blading, cricket, hurling, lacrosse, mountain biking, cycling, bobsleigh, extreme sports e.g. bungee jumping and weightlifting, motorcycling.

The pressure material may also be used in medical applications e.g. for hip protection, head protection for vulnerable people, protective devises to aid recovery from injury and/or orthopaedic devices or in work protection wear e.g. safety gloves, safety footwear, safety clothing.

Another application for the pressure material is in the protection of items or articles into which the pressure material may be incorporated or which may use the pressure material as an encasement, e.g. suitcases, laptop cases, laptop backpacks, camera cases, mobile phone cases, portable music equipment cases, golf clubs, surfboard protection, radio and in packaging for fragile items in transportation, lining of vehicles and crates for transportation. Furthermore, the pressure material may be used in transportation applications such as automobile dashboards, bumpers, and safety equipment in other transport e.g. trains and aeroplanes

In use a plurality of layers of the treated impact protection material may be utilised in order to suit the application for which it is to be used. The layers may be identical or may be a combination of alternative substrates described above or alternatively may be a combination of one or more layers of impact protection material according to the present invention and layers of other materials. Furthermore, the composition described in the present invention may be applied alone to a substrate or may be applied with other suitable materials which do not negatively affect the impact resistance of the treated materials. Examples might include gels, resins and foams or the like.

In accordance with another aspect of the invention, there is provided use of a pressure material as defined above to spread constant or repeated pressure away from one or more pressure points between a user and an item of wear. In accordance with another aspect of the present invention, there is provided a shoe inlay comprising a pressure material as defined above able to spread constant or repeated pressure on the inlay away from one or more pressure points between a use and the inlay.

According to another aspect of this invention, there is provided a shoe comprising a pressure material as defined above.

According to another aspect of the present invention, there is provided a method of treating a symptom of a medical condition caused by one or more pressure points between a use and item of wear, the method comprising the use of a pressure material as defined above to spread constant or repeated pressure away from the one or more pressure points between the use and the item of wear.

The present invention will now be described with reference to the following examples and comparative tests which are not intended to be limiting.

### EXAMPLES

### Example 1

In this example three compositions were prepared with component (B) not cross-linked:
component (A): a vinyl-bond rich SEPS with a styrene content=20% wt % and a hardness=64 shore A according to ISO 868 (Hybrar® 7125 from Kuraray) and
component (B): a HCR (polyvinyldimethylsiloxane) of plasticity from 60-65 MILS (SGM11 from Dow Corning)

### Compositions (Quantities are Given in Parts by Weight):

### Ex 1A: Hybrar® 7125/SGM 11 (85/15)

### Ex 1B: Hybrar® 7125/SGM 11 (70/30)

### Ex 1C: Hybrar® 7125/SGM 11 (60/40)

### Example 2

In this example a further three compositions were prepared with component (B) not cross-linked:
component (A): a vinyl-bond rich SEPS with a styrene content=20% wt % and a hardness=64 shore A according ISO 868 (Hybrar® 7125 from Kuraray) and
component (B): a HCR containing 30% wt of silica of plasticity from 85-140 MILS (Silastic® HS71 from Dow Corning)

### Compositions (Quantities are Given in Parts by Weight):

### Ex 2A: Hybrar®7125/Silastic® HS71 (85/15)

### Ex 2B: Hybrar® 7125/Silastic® HS71 (70/30)

### Ex 2C: Hybrar® 7125/Silastic® HS71 (60/40)

In examples 1 and 2, the compositions are converted into pellets using a twin screw extruder of diameter=25 mm and L/D=48 and water batch cooling system equipped with a granulator.

Extruding conditions: screw speed=200 rpm/output=l5 kg/h/melt temperature=180° C.

Pellets are moulded into 150 mm×150 mm×6 mm (L×I×e) plates using a Krauss Maffei injection moulding machine: moulding temperature: 180° C./mould temperature: 23° C. EXPANCEL® blowing agent can be added at this stage to decrease the density of the moulded part. Thus:
Ex 2D: Hybrar® 7125/Silastic® HS71 (60/40), and 3% of subsequently added EXPANCEL® 092 MB 120.

### Example 3

Six compositions containing a cross-linked silicone as component (B) are made with:

### Ex 3A: Hybrar® 7125/Silastic® HS-71 (60/40)

58.24 wt % of Hybrar® 7125-40 wt % of Silastic® HS71
1.2 wt % of Dow Corning® 7678 (a commercial cross-linker containing at least 3 Si—H bonds per molecule)
0.56 wt % Pt catalyst solution 10%

### Ex 3B: Hybrar® 7125/Silastic® HS-71 (70/30)

68.54 wt % of Hybrar 7125-30 wt % of Silastic® HS71
0.9 wt % of Dow Corning® 7678 cross-linker
0.56 wt % Pt catalyst solution 10%

### Ex 3C: Hybrar® 7125/Silastic® HS-71 (85/15)

84.32 wt % of Hybrar® 7125-15 wt % of Silastic® HS71
0.45 wt % of Dow Corning® 7678 cross-linker
0.28 wt % Pt catalyst solution 10%

### Ex 3D: Hybrar® 7125/SGM 11 (60/40)

58.24 wt % of Hybrar® 7125
40 wt % of SGM 11
1.2 wt % of Dow Corning® 7678 cross-linker
0.56 wt % Pt catalyst solution 10%

### Ex 3E: Hybrar® 7125/SGM 11 (70/30)

68.54 wt % of Hybrar® 7125-30 wt % of SGM 11
0.9 wt % of Dow Corning® 7678 cross-linker
0.56 wt % Pt catalyst solution 10%

### Ex 3F: Hybrar® 7125/SGM 11 (85/15)

84.32 wt % of Hybrar 7125
15 wt % of SGM 11
0.45 wt % of Dow Corning® 7678 cross-linker
0.28 wt % Pt catalyst solution 10%

In Examples 3, the compositions are converted into pellets using a twin screw extruder of diameter=25 mm and L/D=48 (12 barrels) and water batch cooling system equipped with a granulator. Hybrar® 7125 and HCR (Silastic® HS-71 or SGM 11) are introduced respectively in barrel 1 and 2 and thoroughly mixed. The cross-linker is then introduced (on barrel 5) and thoroughly dispersed in the mixture introduced and finally, immediately before the commencement of cure catalyst is introduced through (barrel 8). Component (B) is then cured into the composition via a hydrosilylation cure.

Extruding conditions: screw speed=200 rpm/output=15 kg/h/melt temperature=180° C.

The resulting pellets can then be moulded into required shapes using a suitable press or the like. In the present example the resulting pellets are moulded in 150 mm×150 mm×6 mm (L×I×e) plates using a Krauss Maffei injection moulding machine.

In the event that a foam material is required, a foaming agent, such as by way of example EXPANCEL® 092 MB 120 from the company AKZO NOBEL, may be introduced.

Prepared sheets and foam sheets were then subjected to impact testing. Impact testing was carried out according to EN1621 Parts 1 and 2 “Motorcyclists' protective clothing against mechanical impact”, where a 5 kg weight of specified shape was caused to impact the device held over an anvil of specified shape, such that the impact energy is 50 J. A load cell within the anvil measures the resultant impact force transmitted through the device.

### Example 4

A composition was prepared using the following components, in which the quantities are given in parts by weight.

### Part (i):

65.4 dimethylvinylsiloxy-terminated dimethyl siloxane having a viscosity at 25° C. of 2,000 mPas
4.2 hexamethyldisilazane
1.4 water
29.0 precipitated silica, Degussa FK320DS
0.1 1,3-diethenyl-1,1,3,3-tetramethyldisiloxane platinum complex

The hexamethyldisilazane, water and a portion of the vinylsiloxane were added to a high shear mixer. FK320DS was added incrementally until it was well dispersed. The resulting mixture was then heated to 170° C. under vacuum. The remaining vinylsiloxane and platinum complex were then added.

### Part (ii):

72.5 dimethylvinylsiloxy-terminated dimethyl siloxane having a viscosity at 25° C. of 2,000 mPas
27.0 trimethylsiloxy-terminated dimethyl, methylhydrogen siloxane having a viscosity at 25° C. of 5 mPas and a hydrogen content bonded to silicon of 0.8% w/w
0.5 ethynyl cyclohexanol

The ingredients were mixed until homogeneous, with mild heating (50° C.) which helped to dissolve the ethynyl cyclohexanol.

Parts (i) and (ii) were then mixed in the ratio 10:1 by weight and the blend was designated silicone elastomer 1 (SE1). This mixture was then blended with a polydimethylsiloxane (PDMS) at the required ratio (see herein below for further details of the PDMS).

### Example 5

A composition was prepared using the following components, in which the quantities are given in parts by weight.

### Part (i)

19.8 vinyl functional methyl polysiloxane resin, comprising the Vi(Me)₂SiO_{1/2} unit and SiO_{4/2} unit with Vi group content 3.25% w/w
52.0 dimethylvinylsiloxy-terminated dimethyl siloxane having a viscosity at 25° C. of 2,000 mPas
15.0 dimethylvinylsiloxy-terminated dimethyl siloxane having a viscosity at 25° C. of 55,000 mPas
0.1 1,3-diethenyl-1,1,3,3-tetramethyldisiloxane platinum complex

All ingredients were blended together in a low shear mixer essentially following the procedure of Example 4.

### Part (ii)

7.8 vinyl functional methyl polysiloxane resin, comprising the Vi(Me)₂SiO_{1/2} unit and SiO_{4/2} unit with Vi group content 3.25% w/w
61.0 trimethylsiloxy-terminated dimethyl, methylhydrogen siloxane having a viscosity at 25° C. of 5 mPas and a hydrogen content bonded to silicon of 0.8% w/w
24.3 dimethylvinylsiloxy-terminated dimethyl siloxane having a viscosity at 25° C. of 2,000 mPas
0.5 ethynyl cyclohexanol

All ingredients were blended together in a low shear mixer essentially following the procedure of Example 4.

Parts (i) and (ii) were then mixed in the ratio 10:1 by weight and the blend was designated silicone elastomer 2 (SE2). This mixture was then blended with PDMS at the required ratio (see below).

### Example 6

A composition was prepared using the following components, in which the quantities are given in parts by weight.

### Part (i)

12.0 dimethylvinylsiloxy-terminated dimethyl siloxane having a viscosity at 25° C. of 55,000 mPas
52.0 dimethylvinylsiloxy-terminated dimethyl siloxane having a viscosity at 25° C. of 2,000 mPas
16.8 vinyl functional methyl polysiloxane resin, comprising the Vi(Me)₂SiO_{1/2} unit and SiO_{4/2} unit with Vi group content 3.25% w/w
0.25 water
0.82 hexamethyldisilazane
4.9 fumed silica, Cabot MS75
0.2 1,3-diethenyl-1,1,3,3-tetramethyldisiloxane platinum complex

A majority of the vinyl polymers, silica, water and treating agents were combined. The mixture was then heated and stripped under vacuum. The remaining polymer was then added.

### Part (ii)

12.0 dimethylvinylsiloxy-terminated dimethyl siloxane having a viscosity at 25° C. of 55,000 mPas
36.7 dimethylvinylsiloxy-terminated dimethyl siloxane having a viscosity at 25° C. of 2,000 mPas
0.25 water
0.82 hexamethyldisilazane
4.9 fumed silica, Cabot MS75
11.8 vinyl functional methyl polysiloxane resin, comprising the Vi(Me)₂SiO_{1/2} unit and SiO_{4/2} unit with Vi group content 3.25% w/w
9.3 trimethylsiloxy-terminated dimethyl, methylhydrogen siloxane having a viscosity at 25° C. of 5 mPas and a hydrogen content bonded to silicon of 0.8% w/w
1.8 trimethylsiloxy terminated dimethyl, methylhydrogen siloxane having a viscosity at 25° C. of 30 mPas and a hydrogen content bonded to silicon of 1.6% w/w
0.1 ethynyl cyclohexanol

A majority of the vinyl polymers, silica, water and treating agents were combined. The mixture was then heated and stripped under vacuum. The remaining polymer was then added.

Parts (i) and (ii) were then mixed in the ratio 10:1 by weight and the blend was designated silicone elastomer 3 (SE3). This mixture was then blended with a PDMS at the required ratio (see below).

### Example 7

The mixtures produced in Examples 4-6 were impregnated at different ratios into various substrates, namely:

a 5 mm thick spacer fabric of 590 g/m²
a 7 mm thick spacer fabric of 570 g/m²
a 8 mm non-woven fabric of 500 g/m²

Suppliers of spacer fabrics include: Baltex, UK; CIMA, Spain; Dafa, PRC; Heathcoat, UK; Mueller, Germany; and Scott & Fyffe, UK. Suppliers of non-woven fabrics include Captiqs, Belgium; Danweb, Denmark; Ecotextil, Czech Republic; Freudenberg, Germany; JSC Neaustima, Lithuania; Sandler, Germany; and Ziegler, Germany.

The fabric was then heat treated at 180° C. for 10 minutes to cure the silicone. The handle of the impregnated fabric was soft and flexible with good resilience, but without excessive impacter bounce.

### Test

### Example 1

29 Year old marathon runner. Currently running 80 miles per week. Fore foot runner. 3 month history of second metatarsal head pain. Medical examination including X-ray are reported as clear. Dynamic foot scan suggests neutral foot type with high pressure evident over second metatarsal head. Patient is found to have a very long second metatarsal when compared to the 1st metatarsal, resulting in prolonged loading of the second metatarsal in gait, and increased trauma when fore foot running.

### Intervention:

Shoe liner removed, and 3 mm pressure material (made as per Example 2D hereinabove in 3 mm form) flat beds added to patient's own running shoes. Shoe liner replaced. Difference in metatarsal pressure points shown in table below.

### Outcome:

Patient attended for a review appointment 2 weeks following issue of pressure material, reporting total resolution of symptoms. Patient is back to full training with no discomfort and no further intervention indicated. Patient Discharge

Measured at maximum transmitted pressure (N/cm2)

| | Left without | Left with |
|---|---|---|
| | pressure material | pressure material |
| Toe 1 | 0.5 | 0.5 |
| Toe 2-5 | 0 | 0 |
| Metatarsal 1 | 10 | 7 |
| Metatarsal 2 | 24 | 10 |
| Metatarsal 3 | 16 | 7 |
| Metatarsal 4 | 11 | 12 |
| Metatarsal 5 | 4 | 2 |
| Midfoot | 7 | 9 |
| Maximum plantar pressure | 24 | 12.5 |
| | Right without | Right with |
| | pressure material | pressure material |
| Toe 1 | 8 | 5 |
| Toe 2-5 | 6 | 9 |
| Metatarsal 1 | 16 | 10 |
| Metatarsal 2 | 22 | 9 |
| Metatarsal 3 | 27 | 14.9 |
| Metatarsal 4 | 4 | 2 |
| Metatarsal 5 | 4 | 4 |
| Midfoot | 4 | 2 |
| Maximum plantar pressure | 26.8 | 14.9 |

1. A method of spreading constant or repeated pressure away from one or more pressure points between a user and an item of wear wherein the item of wear comprises a pressure material being either:
(i) a thermoplastic material having the composition comprising a mixture of;
(a) component (A) an organic thermoplastic elastomer having a hardness below 80 shore A measured at 23° C. (ISO 868); and
(b) component (B) which is a non cross-linked and substantially non reactive silicone polymer or a cross-linked silicone polymer, with the exclusion of borated silicone polymers exhibiting dilatant properties; or
(ii) a flexible material having the composition comprising a mixture of;
(c) an elastomeric material having a modulus at 100% elongation of 0.1-10 MPa; and
(d) 5-80% by weight, based on the total weight of the composition of a non-reactive silicone fluid having a viscosity of 1,000-3,000,000 mPas at 25° C.; or
(iii) both (i) and (ii).

2. A method as claimed in claim 1, wherein the item of wear is a shoe inlay.

3. A method as claimed in claim 1 wherein the item of wear is a shoe.

4. A method as claimed in claim 3 wherein the pressure material is a layer of the sole of the shoe.

5. A method as claimed in claim 1 comprising spreading constant or repeated pressure away from at least two pressure points.

6. A method as claimed in claim 5 wherein the at least two pressure points are in a foot.

7. A method as claimed in claim 6 wherein at least one pressure point is or involves a metatarsal.

8. A method as claimed in claim 1, wherein the pressure material is laminated with one or more other wear layers.

9. A method as claimed in as claimed in claim 1, wherein the pressure material is in the form of a sheet.

10. A method as claimed in claim 1, wherein the pressure material is foamed.

11. A method as claimed in claim 1, wherein the pressure material is a thermoplastic material and component (B) comprises:
i a silicone fluid with a Brookfield viscosity from 1,000 mPa·s to 3,000,000 of mPas at 25° C. (all viscosities, where possible, unless otherwise mentioned, are measured by Brookfield Rotational Viscometer, Model DVIII, Spindle CP52 at 0.5 rpm at 25° C.);
ii a silicone gum with a molecular weight from 50,000 g/mol to 700,000 g/mol, or
iii a silicone preparation as liquid silicone rubber or high consistency rubber with no cross-linker and catalyst.

12. (canceled)

13. A method of treating one or more of the group comprising:
metatarsalgia; sesamoiditis/fractures; diabetes; stress fractures; shin splints; knee pain; gout; OA; heel spurs; supinated/pes cavus foot types resulting in high plantar pressures; and plantar ulcer, (where there may be some ischaemia caused by circulatory impairment) comprising
adding a pressure material as defined in claim 1, in the shoe or shoes of the person with such medical condition.

14. A shoe inlay comprising a pressure material as defined in claim 1, which is able to spread constant or repeated pressure away from one or more pressure points between a user and the inlay.

15. A shoe comprising a pressure material as defined in claim 1 which is able to spread constant or repeated pressure away from one or more pressure points between a user and the shoe.

16. A method as claimed in claim 1, wherein the pressure material is (ii) a flexible material having the composition comprising a mixture of;
(c) an elastomeric material having a modulus at 100% elongation of 0.1-10 MPa; and
(d) 5-80% by weight, based on the total weight of the composition of a non-reactive silicone fluid having a viscosity of 1,000-3,000,000 mPas at 25° C.

17. A method as claimed in claim 16, wherein the pressure material is in the form of a sheet.

18. A method as claimed in claim 16, wherein the pressure material is foamed.

19. A shoe inlay comprising a pressure material as defined in claim 17 which is able to spread constant or repeated pressure away from one or more pressure points between a user and the inlay.

20. A shoe inlay comprising a pressure material as defined in claim 18 which is able to spread constant or repeated pressure away from one or more pressure points between a user and the inlay.

21. A method as claimed in claim 16, wherein the pressure material is laminated with one or more other wear layers.
